(19) United States
(12) Patent Application Publication
(10) Pub. No.: US 2005/0203593 A1
(43) Pub. Date: Sep. 15, 2005

(54) Method for dermatology therapies in combination with low level laser treatments

(76) Inventors: Steven C. Shanks, Moon, AZ (US); Richard Amy, Fallbrook, CA (US); Jeffrey M. Nelson, (US); Holla'e Ploof-Mnatzaganian, Tucson, AZ (US)

Correspondence Address: ETHERTON LAW GROUP, LLC, 5555 E. VAN BUREN STREET, SUITE 100, PHOENIX, AZ, 85008, US

(21) Appl. No.: 10/973,608

(22) Filed: Oct. 25, 2004

Related U.S. Application Data
(60) Provisional application No. 60/514,162, filed on Oct. 24, 2003.

(51) Int. Cl. A61N 5/06

(52) U.S. Cl. 607/89; 606/9; 606/20; 606/32

(57) ABSTRACT

The present invention is a method for promoting faster wound healing and alleviating pain during and after various dermatology-related treatments. The method comprises using low level laser therapy in conjunction with dermatological treatments including intense pulsed light, radio frequency, dermabrasion, microdermabrasion, chemabrasion, chemical peels, ablative lasers, and cryogenics.

### CROSS-REFERENCE TO RELATED APPLICATIONS

This application claims the benefit of co-pending provisional application No. 60/514,162 filed Oct. 24, 2003.

### BACKGROUND

This invention relates generally to an improved method for dermatological surgery and therapy that promotes faster wound healing and alleviates pain. This invention relates specifically to the application of low-level laser in conjunction with dermatological treatments for skin restoration.

Skin is made up of two layers, the visible outer layer called the epidermis, and the deeper layer called the dermis. The main structural component of the dermis is a protein called collagen, which provides the skin its strength. Undesirable conditions of the skin include wrinkles; rosacea; enlarged pores; sun damage; actinic keratoses; actinic chelitis; acne vulgaris; brown spots such as age-spots and freckles; hyper- and hypo-pigmentation, broken blood vessels; vascular pigmented lesions, including telangiectasias (“spider veins”) and hemangiomas; scars, including hypertrophic scars, as a result of acne, trauma, burns and surgery; unwanted hair or tattoos.

Numerous treatments have been developed to improve the appearance of the skin. During the past decade the trend has been to seek procedures for all skin types, including darker skinned patients, which heretofore have suffered complications such as keloiding, hyper- or hypo-pigmentation. While chemical peels (“chemabrasion”), dermabrasion and microdermabrasion are still popular courses of treatment in many physicians' offices, more doctors are utilizing ablative laser and light source treatments. Other methods used to improve the skin include invasive dermatological treatments in which portions of the skin are damaged or removed, including intense pulsed light (“IPL”), radio frequency (“RF”), ablative lasers, and cryogenics.

To a greater or lesser degree, each of these therapies produces some discomfort and considerable downtime for recovery. Certain types of conditions are difficult to correct without further destruction of the surrounding skin structures. Furthermore, while the end results can be beautiful, each treatment causes, to a greater or lesser degree, damage that causes the patient's skin to be unsightly for a period of time until it heals. Patients express concerns with pain during and post-recovery, as well as the length of the recover periods. Less invasive approaches often require repeat treatments over a period of five or more months. Even these less aggressive treatments may cause the skin to become slightly pink or puffy for a day or so. The more aggressive treatments, such as with the ablative lasers, cause severe swelling, redness, crusting or scabbing, bumps and blisters on or around the treated area, and sometimes scabbing. Extended hearling periods are almost always involved, causing interruption in in normal activities and, many times, loss of work. Patients benefit when less pain is experienced, reducing medication levels as well as minimizing post-procedure bruising while increasing tissue perfusion. It is desirable to reduce the amount of pain and healing time.

Low energy laser therapy (LLLT) is used in the treatment of a broad range of conditions. LLLT improves wound healing, reduces edema, and relieves pain of various etiologies, including successful application post-operatively to liposuction to reduce inflammation and pain. LLLT is also used during liposuction procedures to facilitate removal of fat by causing intracellular fat to be released into the interstice. It is also used in the treatment and repair of injured muscles and tendons.

LLLT utilizes low level laser energy, that is, the treatment has a dose rate that causes no immediate detectable temperature rise of the treated tissue and no macroscopically visible changes in tissue structure. Consequently, the treated and surrounding tissue is not heated and is not damaged. There are a number of variables in laser therapy including the wavelength of the laser beam, the area impinged by the laser beam, laser energy, pulse width, treatment duration and tissue characteristics. The success of each therapy depends on the relationship and combination of these variables. For example, liposuction may be facilitated with one regimen utilizing a given wavelength and treatment duration, whereas pain may be treated with a regimen utilizing a different wavelength and treatment duration, and inflammation a third regimen. Specific devices are known in the art for each type of therapy.

It would be desirable to combining the low level laser therapy with dermatological therapies to enable the patent to experience less pain and heal faster. This can be extremely useful for procedures that require multiple treatments in order to get the desired results. For example, if the patient has less pain, then the practitioner can be more aggressive with subsequent treatments and, with faster healing, the interval between treatments will be shorter. The patient will thus achieve the desired results faster.

### SUMMARY OF THE INVENTION

The present invention is a method for promoting faster wound healing and alleviating pain during and after various dermatology-related treatments. The method comprises using low level laser therapy in conjunction with dermatological treatments including intense pulsed light, radio frequency, dermabrasion, microdermabrasion, chemabrasion, chemical peels, ablative lasers, and cryogenics.

### BRIEF DESCRIPTION OF THE DRAWINGS

FIG. 1 illustrates the method of the preferred embodiment.

FIG. 2 is an electrical schematic illustration of a preferred embodiment of the laser used in the present invention.

FIG. 3 is a schematic view of the optical arrangement of the linear spot shape of the preferred embodiment.

FIG. 4 is a schematic view of the optical arrangement of an alternate spot shape.

FIG. 5 is a schematic illustration of application of low-level laser radiation on a patient's face using the preferred embodiment of the present invention.

### DETAILED DESCRIPTION OF THE INVENTION

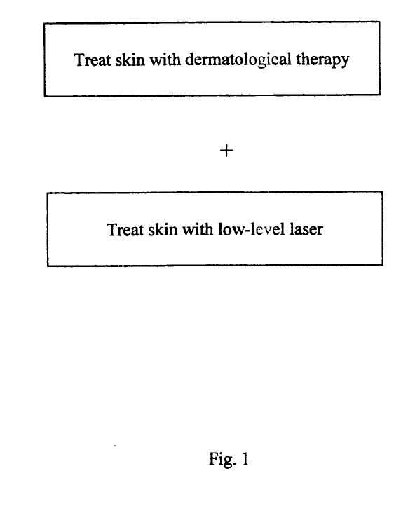

The present invention is a method for treating skin comprising treating skin with one or more dermatological therapies and low-level laser light therapy. See FIG. 1. The laser light therapy is applied before, during, or after—or a combination of all three—the dermatological therapy, depending on the type of skin defect and dermatological therapy used. Multiple types of dermatological treatments can be combined with the low-level laser therapy.

In one embodiment, intense pulsed light (“IPL”) therapy, also known as the photofacial, is applied to the patient. IPL uses high levels of broad spectrum, incoherent light power in millisecond bursts to destroy specific layers deep in the skin, leaving the surface undamaged. Preferably a krypton or xenon flashlamp emitting 500-1400 nm energy in pulses is employed, for example those available commercially from DermaMed™ and Lumenis™. Low-level laser light therapy is applied during or after the IPL therapy, preferably using the laser device described in detail below.

In another embodiment, the patient is treated with thermoplastic contouring. This therapy uses radio frequencies (“RF”) to induce resistive heating of a desired area of collagen under the surface of the skin. Heating the collagen to critical temperatures initiates a natural biological reaction that causes the collagen to contract and thicken. This process is called denaturation. To prevent the surface skin from heating, at least one commercial device employs cryogenic contact cooling prior to, during, and after RF delivery, to create a reverse thermal gradient whereby the greatest heating is deep in the tissue while the outermost layer, the epidermis, is not heated. This product is available commercially as a Thermacool TC™ system from Thermage™. Low-level laser light therapy is applied during or after the RF therapy, preferably using the laser device described in more detail below.

In yet another embodiment, dermabrasion is used to treat the patient. Dermabrasion removes the skin surface by sanding or wire-brushing off some of the outer skin layer by various means so as to create a wound which is superficial enough to re-grow normally and deep enough to eliminate a pathology like tattoo or acne scar. Dermabraders are known in the art and are available commercially from several sources. Low-level laser light therapy is applied during or after the dermabrasion therapy, preferably using the laser device described in detail below.

Similarly, a patient can be treated by microdermabrasion, also known as the crystal or power peel. This dermatological treatment blasts the top layer of skin with an extremely fine mixture of crystals, removing the dead cells. Typically aluminum-oxide crystals are used. Dermabraders and microdermabraders are available commercially from several sources including DermaMed™ (the MegaPeel™) and General Project™ (Duo-peel™). Low-level laser light therapy is applied during or after the microdermabrasion therapy, preferably using the laser device described in detail below.

Chemabrasion uses chemicals to remove the outer layers of skin. The chemical normally used is trichloracetic acid (TCA) for more superficial lines and blemishes or phenol for deeper wrinkles. The chemical effect may be accelerated or multiplied by the application of non-laser light, such as the Blu-u Light™ treatment offered by Levulan™. In the Blu-u Light treatment, a topical solution of aminolevulinic acid HCl 20% is applied, followed by exposure of the skin to blue non-laser light. Low-level laser light therapy is applied during or after the chemabrasion therapy, preferably using the laser device described in detail below.

Cryogenic therapy uses a very cold substance, typically liquid carbon dioxide, to freeze a layer of skin and kill the cells. Low-level laser light therapy is applied during or after the cryo therapy, preferably using the laser device described in detail below.

Ablative lasers, such as CO₂, Nd:YAG, ruby, Er:YAG, Nd:YAP, and long-pulsed Alexandrite lasers, are used to destroy or damage skins cells. These high-powered lasers destroy the skin cells by raising the temperature of the cells, essentially burning them. Such lasers are available commercially from various sources including Symedex™ and Lumenis™. Low-level laser light therapy is applied during or after the ablative laser therapy, preferably using the low-level laser device described in detail below.

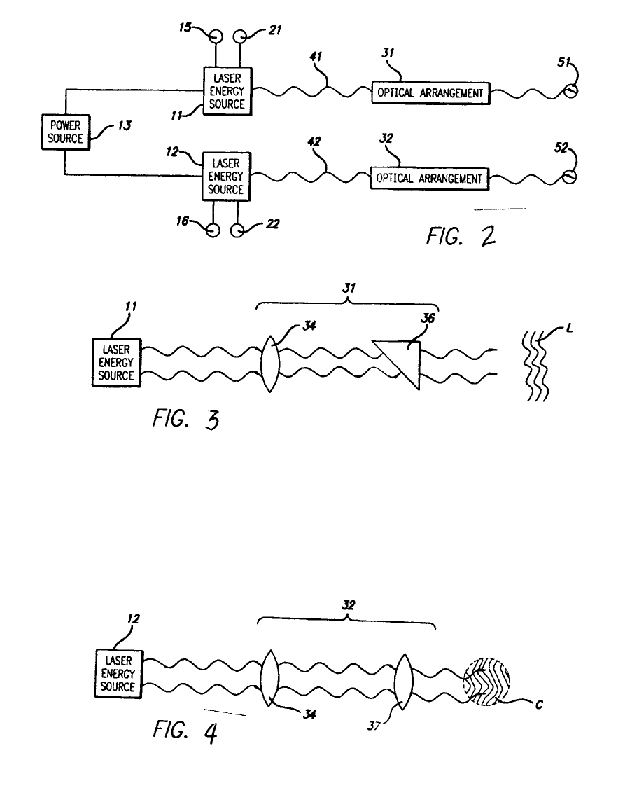

FIG. 2 shows the laser device used herein in which a first laser energy source 11 and a second energy source 12 are connected to a power source 13. The power source preferably provides direct current, such as that provided by a battery, but may instead provide alternating current such as that provided by conventional building current that is then converted to direct current. Separate control means 15, 16 are connected to the laser energy sources 11, 12 respectively and act as on/off switches to control the period of time the laser light is generated. These laser energy sources can be energized independently or simultaneously which, throughout this specification, refers to acts occurring at generally at the same time.

Laser energy sources are known in the art for use in low-level laser therapy. They include Helium-Neon lasers having a 632 nm wavelength and semiconductor diode lasers with a broad range of wavelengths between 600-800 nm. The laser energy sources in the preferred embodiment are two semiconductor laser diodes that produce light in the red range of the visible spectrum, having a wavelength of about 635 nm. Other suitable wavelengths are used for other particular applications. While many LLLT regimen include visible laser light, it is advantageous to utilize at least one laser beam in the visible/UV energy spectrum so that the operator can see the laser light as it impinges the patent's body and the area treated can be easily defined. Solid state and tunable semiconductor laser diodes may also be employed to achieve the desired wavelength.

Different therapy regimens require diodes of different wattages. The preferred laser diodes use less than one watt of power each to simultaneously facilitate liposuction, treat post-operative inflammation, and post-operative pain. Diodes of various other wattages may also be employed to achieve the desired laser energy for the given regimen.

Control means 21, 22 are connected to the laser energy sources 11, 12, respectively, to form a control circuit that controls the duration of each pulse of laser light emitted, referred to herein as the pulse width. When there are no pulses, a continuous beam of laser light is generated. Pulse widths from 0 to 100,000 Hz may be employed to achieve the desired effect on the patient's tissue. The goal for LLLT regimen is to deliver laser energy to the target tissue utilizing a pulse width short enough to sufficiently energize the targeted tissue and avoid thermal damage to adjacent tissue.

Each laser beam 41, 42 exits the laser and is shone through optical arrangements 31, 32, respectively, that produce beam spots 51, 52 respectively of certain shapes. The beam spot is the cross-sectional shape and size of the emitted beam as it exits the optical arrangement. For example, a laser beam of circular cross-section creates a circular beam spot as the laser light impinges the patient's skin. If the laser light emitted is in the visible range, a circular spot can be seen on the patient's skin of substantially the same diameter as the laser beam emitted from the optics arrangement. In the preferred embodiment, the first laser beam is passed through an optical arrangement that generates a beam of substantially linear cross-section, resulting in a line of laser light seen on the patient's skin. The second laser passes through an optical arrangement that generates a beam of circular cross-section, resulting in a circular spot shape as seen on the patient's skin.

As shown in FIG. 3 the first optical arrangement 31 of the preferred device 10 includes a collimating lens 34 and a line generating prism 36. The collimating lens 34 and the line generating prism 36 are disposed in serial relation to the laser energy source 11. The collimating lens 34 and the line generating prism 36 receive and transform the generated beam of laser light into the line of laser light L. As an alternative, a suitable electrical or mechanical arrangement could be substituted for the optical arrangement 31.

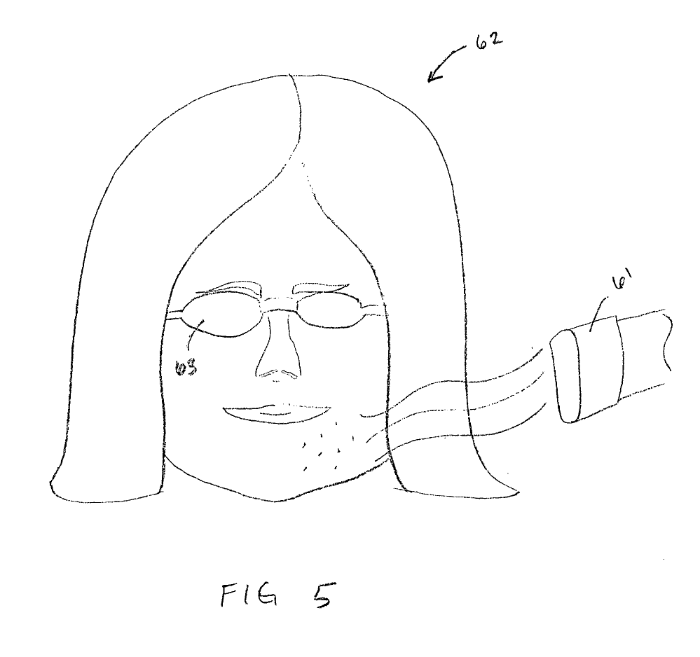

As shown in FIG. 4 the second optical arrangement 32 of the preferred device 10 includes a collimating lens 34. As with the first optical arrangement, the collimating lens 34 is disposed in serial relation to the laser energy source 12. The collimating lens 34 receives and transforms the generated beam of laser light into a circular beam spot of laser light C. As an alternative, a suitable electrical or mechanical arrangement could be substituted for the optical arrangement 32 to achieve a desired spot shape.

The device may utilize as many lasers and optical arrangements as necessary to obtain the desired emissions and spot shapes. For example, the device may employ two laser diodes each with a collimating lens, such that two substantially circular spot shapes are achieved. Or, for example, the device may employ two laser diodes each with an optical arrangement such that two substantially linear spot shapes are achieved. Or, in another example, more than two lasers may be used and optical arrangements aligned such that two or more of the laser beams have substantially similar spot shapes and are co-incident where they impinge the patient's skin.

In order to direct the laser light to the desired area on a patient, the laser light is emitted from a lightweight, hand-held pointer referred to herein as a wand 61. See FIG. 5 which shows the laser light applied to a patient wearing goggles for eye protection. The wand 61 is preferably an elongated hollow tube defining an interior cavity which is shaped to be easily retained in a user's hand. In the preferred embodiment the laser energy sources 11, 12 are mounted in the wand's interior cavity, although the laser energy sources could be remotely located and the laser light conducted by fiber optics to the wand. The wand may take on any shape that enables the laser light to be directed as needed such as tubular, T-shaped, substantially spherical, or rectangular (like a television remote control device).

There are a number of variables in the present method including, but not limited to, the type or types of dermatological treatment used, duration of the treatment, wavelength of the laser beam, the area impinged by the laser beam, laser energy, pulse width, treatment duration and tissue characteristics. The success of each therapy depends on the relationship and combination of these variables. The following are specific examples of the method of the present invention, but it is not a comprehensive listening of the potential combinations and does not exhaust the types of therapies described by the present method.

### EXAMPLE 1

### Hypertrophic Burn Treatment

Hypertrophic scars that are woody and elevated are treated in consort with laser or IPL by triple stacking through the rigid scar. The scar chars during treatment, which flattens the elevated tissue. Immediate use of LLLT reduces inflammation which can contribute to further keloid formations. LLLT further prevents water influx into the scar which can cause expansion of the flattened tissue. While normally five to seven treatments are needed to ablate a hypertrophic scar, the patient achieves a superior cosmetic appearance, significant flattening, softening reduced itching and burning sensations, as well as rapid healing in two or three treatments.

### EXAMPLE 2

### Actinic Chelitis Treatment

Male patent presented with pre-operative diagnosis of squamous cell carcinoma of the lower lateral right lip. A wide local excision was made excising residual squamous cell carcinoma with frozen section control and tissue rearrangement. Pathology results showed superficially invasive squamous cell carcinoma with multiple sites of carcinoma in situ or other premalignant lesions. Patient was then scheduled for total lip excision. An alternative course of treatment was given instead, using a combination of LLLT, IPL, ALA and photodynamic therapy (PDT). In the first of two treatments, the area was pre-treated with LLLT to avoid spontaneous blistering of the mucosal vascular lip tissue, which would otherwise have eliminated the application of ALA, which cannot be applied to broken tissue. The ALA was applied to the lip and allowed to incubate for 8 hours. The ALA was cleansed from the site and Blu-U-Light was applied in a phase called activation. LLLT was applied prior to activation for three minutes at settings of 19-29-39-18 to reduce acute inflammatory response. LLLT was also applied during activation to promote more rapid healing and a to eliminate blistering or water influx into the scar. In the second treatment, the area was again pre-treated with LLLT. The ALA was applied to the lip and allowed to incubate for 18 hours. The ALA was cleansed from the site and Blu-U-Light at 417 nm was applied. LLLT was applied prior to activation for three minutes at settings of 19-29-39-18.

### EXAMPLE 3

### Scar Treatment Using a Combination of Cryo, IPL and LLLT Treatments

A keloid scar was pre-treated with LLLT. The scar was then treated with IPL triple-stacked pulsing to burn through the elevated scar. The tissue charred, with an immediate flattening of the tissue. Immediately following the charring response, the scar was treated with LLLT. The scar was also treated to reduce heat retention, preferably with a cryogenic treatment at −30 using a Zimmer Elektromedizin Cryo-5 unit. Tissue treated as such virtually eliminates any additional keloid formation or a worsening of the hypertrophic scar. No blistering is observed. ALA was applied to the scar and allowed to incubate for 8 hours. The ALA was cleansed from the site and activated with Blu-light, followed by a less aggressive IPL treatment with double-stacked pulsing. After activation, from 2-10 minutes, the tissue was again cooled for several minutes. The scars remained flattened and no water influxed into the scar so that conventional long-term compression bandaging was not needed. LLLT was applied once a day for 3-4 days.

### EXAMPLE 4

### Scar Treatment Using a Combination of Cryo, IPL and LLLT Treatments

A hypertrophic scar was treated with cryogenic treatment at −30 using a Zimmer Elektromedizin Cryo-5 unit. The cryo treatment continues while three continuous IPL pulses at 30 j/cm2 were applied to the scar. Charring was observed on the edges of the scar along with visually flattened tissue. LLLT was applied after the IPL treatment. The scar appeared black the next day. Two treatments were required to remove the scar, compared to the four to six treatment normally required when no LLLT is used.

### EXAMPLE 5

Scar Treatment Using a Combination of IPL with Blue Light and LLLT Treatments

The skin is treated with four brisk acetone scrubs to lift traces of oil residue and bring the skin to pin-point bleeding. The skin is treated with a single IPL pulse at 27 j/cm2, followed by an application of ALA and LLLT. After 16 hours, the ALA is washed from the skin and the skin is treated with a second IPL treatment at 15 j/cm2, followed by LLLT. The ALA is activated with the Dusa BLU-U-Light PDT Illuminator in 4 minute increments simultaneously with LLLT.

While there has been illustrated and described what is at present considered to be a preferred embodiment of the present invention, it will be understood by those skilled in the art that various changes and modifications may be made, and equivalents may be substituted for elements thereof without departing from the true scope of the invention. Therefore, it is intended that this invention not be limited to the particular embodiment disclosed as the best mode contemplated for carrying out the invention, but that the invention will include all embodiments falling within the scope of the appended claims.

1. A method for treating skin comprising:
a) treating skin with a dermatological therapy to repair defects; and
b) applying low-level laser light to the skin that is treated.

2. The method of claim 1 in which treating the skin is invasive.

3. The method of claim 2 in which treating the dermatological therapy is one or more of:
a) intense pulsed light;
b) radio frequency;
c) dermabrasion;
d) microdermabrasion;
e) chemabrasion;
f) chemical peel;
g) ablative laser; or
h) cryogenics.

4. The method of claim 1 in which applying the low-level laser light occurs prior to the treating skin with a dermatological treatment.

5. The method of claim 1 in which applying the low-level laser light occurs during the treating skin with a dermatological treatment.

6. The method of claim 1 in which applying the low-level laser light occurs after the treating skin with a dermatological treatment.

7. The method of claim 1 in which applying the low-level laser light occurs prior to and during the treating skin with a dermatological treatment.

8. The method of claim 1 in which applying the low-level laser light occurs during and after the treating skin with a dermatological treatment.

9. The method of claim 1 in which applying the low-level laser light occurs prior to and after the treating skin with a dermatological treatment.

10. The method of claim 1 in which applying the low-level laser light occurs prior to, during and after the treating skin with a dermatological treatment.

11. The method of claim 1 in which applying the low-level laser light is accomplished with a laser device comprising:
a) a plurality of laser energy sources for generating a plurality of laser beams;
b) a wand from which the laser beams emit, the wand being capable of being retained in a hand of a user and freely moved relative to the surface of the skin of a patient; and
c) an optical arrangement attached to the wand for receiving the laser beams and for transforming each of the laser beams into a desired spot shape.

12. The method according to claim 11 wherein at least two of the laser beams are emitted simultaneously.

13. The method according to claim 11 further comprising a controller for independently controlling the generation of laser energy by each of the plurality of laser energy sources.

14. The method according to claim 11 wherein each of the laser energy sources is less than one watt.

15. The method according to claim 11 wherein at least one of the laser energy sources is a semiconductor diode.

16. The method according to claim 11 wherein the laser energy source generates a laser beam having a wavelength in the visible range.

17. The method according to claim 11 wherein at least one of the spot shapes is substantially linear.

18. The method according to claim 11 further comprising a first laser beam having a first spot shape and a second laser beam having a second spot shape wherein the first spot shape is substantially linear and the second spot shape is circular.

19. The method according to claim 11 further comprising a control circuit for controlling the pulse width of each laser beam.

20. The method according to claim 11 wherein the pulse width of at least one of the laser beams is such that the laser light emitted is substantially continuous.
